(19) United States
(12) Patent Application Publication
(10) Pub. No.: US 2025/0206696 A1
(43) Pub. Date: Jun. 26, 2025

(54) PROCESS FOR PRODUCING L-CARNITINE

(71) Applicants: Hubei Xujie Pharmaceutical Co. Ltd, Jingmen City (CN); Carnitech LLC, North Brunswick, NJ (US)

(72) Inventors: Songzhou Hu, Princeton, NJ (US); Lijun Deng, Hubei (CN); Wen Yu, Hubei (CN); Zhen Song, Shanghai (CN)

(21) Appl. No.: 18/393,246

(22) Filed: Dec. 21, 2023

(51) Int. Cl. C07C 227/02; C07C 213/02; C07C 213/10; C07C 253/16

(52) U.S. Cl. CPC C07C 227/02; C07C 213/02; C07C 213/10; C07C 253/16

(57) ABSTRACT

There is disclosed an improved process for the production of L-carnitine, comprising the steps of: (a) reacting(S)-epichlorohydrin with trimethylamine hydrochloride in an organic solvent to yield L-3-chloro-2-hydroxylpropyl trimethylammonium chloride; (b) reacting the L-3-chloro-2-hydroxypropyl trimethylammonium chloride of step (a) with a source of cyanide to yield L-carnitinenitrile chloride; and (c) converting the L-carnitinenitrile chloride of step (b) to L-carnitine.

### TECHNICAL FIELD

The invention relates to an improved process for the production of L-carnitine, in particular, it relates to an improved process for carrying out the reaction of optically active epichlorohydrin with trimethylamine salt in an organic solvent to minimize the racemization in aqueous solution.

### BACKGROUND OF THE INVENTION

L-carnitine is a naturally occurring quaternary ammonium acid involved in metabolism in most mammals, plants, and some bacteria. L-carnitine is also called vitamin BT and has a molecular structure of formula (I). It plays a critical role in energy production by transporting long-chain fatty acids into mitochondria so they can be oxidized to produce energy. L-carnitine also plays an important role in the regulation of metabolic pathways involved in skeletal muscle protein balance. Furthermore, L-carnitine acts as an anti-oxidant and as an anti-inflammatory compound. Therefore, L-carnitine finds wide applications as a nutritional supplement and feed additive.

There are many methods for the synthesis of L-carnitine, but only two methods are being used commercially.

The first process is the fermentative oxidation of gamma-butyrobetaine as depicted in the following reaction:

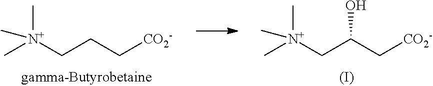

The second process starts from epichlorohydrin and accounts for the majority of L-carnitine produced. The reactions in the second process are described in the following scheme:

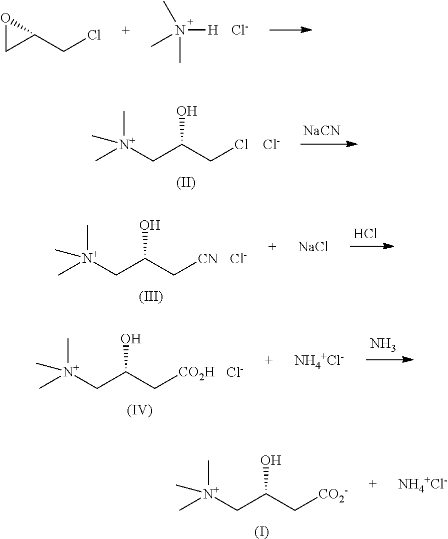

Racemic epichlorohydrin can be efficiently resolved into (S)-epichlorohydrin by using a Jacobsen Co(Salen) catalyst in high yield and high optical purity. (S)-epichlorohydrin is then reacted with trimethylamine hydrochloride to form L-3-chloro-2-hydroxyporpyl trimethylammonium chloride (II), which is subsequently reacted with sodium cyanide to form L-carnitinenitrile chloride (III). These two steps can be carried out in a one-pot process without isolating the intermediate (II) in water. After isolation and purification, L-carnitinenitrile chloride is hydrolyzed in concentrated hydrochloric acid to yield L-carnitine and ammonium chloride. L-carnitine is finally isolated from this solution comprised of L-carnitine, excess hydrochloric acid, and ammonium chloride. The process according to this reaction scheme has been described in U.S. Pat. No. 9,096,493.

The reaction of epichlorohydrin with trimethylamine hydrochloride to form 3-chloro-2-hydroxyltrimethylammonium chloride is well known. In addition to the production of 3-chloro-2-hydroxyltrimethylammonium chloride, also known are several byproducts, such as 1,3-dichloro-2-propanol, 3-chloropropanediol, epoxy byproduct, and bis(trimethylammonium chloride)-2-propanol, i.e., the diquarternary salt.

U.S. Pat. No. 5,077,435 discloses a process to carry out the reaction of epichlorohydrin and trimethylamine hydrochloride in the presence of 1,3-dichloro-2-propanol as a cosolvent to reduce the formation of diquarternary salt, epoxy byproduct, and dichloropropanol. U.S. Pat. No. 5,463,127 further discloses that the reaction of epichlorohydrin and trimethylamine hydrochloride should be carried out at an initial pH of at least 8 by adding trimethylamine. U.S. Pat. No. 4,602,110 discloses a method to purify the product by crystallization from water-soluble alcohols.

U.S. Pat. No. 4,450,295 discloses a process of producing anhydrous trimethylamine chloride and then reacting it with epichlorohydrin to produce anhydrous 3-chloro-2-hydroxypropyl trimethylammonium chloride that is practically free of diquarternary bis(trimethylammonium chloride)-2-propanol.

U.S. Pat. No. 4,594,452 discloses a process for producing solid anhydrous 3-chloro-2-hydroxypropyl trimethylammonium chloride by carrying out the reaction of epichlorohydrin and trimethylamine hydrochloride in an organic solvent, which is a solvent for the two reactants, but a non-solvent for the product. Chloroform was disclosed to be a particularly suitable solvent.

JPH03287567 discloses in detail the reaction of chiral epichlorohydrin with trimethylamine hydrochloride to produce an optically active 3-chloro-2-hydroxylpropyl trimethylammonium chloride. When(S)-epichlorohydrin was reacted with an aqueous solution of trimethylamine hydrochloride, a solid product was obtained in a yield of 98.1% on the basis of trimethylamine. This crude product had a specific rotation of [α]_{D}²⁵=−23.4° (c=1.0, H₂O) and was purified by recrystallization twice in water to obtain a pure product, L-3-chloro-2-hydroxylpropyl trimethylammonium chloride, which has a specific rotation of [α]_{D}²⁵=−30.1° (c=1.0, H₂O) and a melting point of 215.5° C. When (R)-epichlorohydrin was used in the same reaction, a product was obtained in a yield of 96.8% with a specific rotation of [α]_{D}²⁵=+23.2° (c=1.0, H₂O). Recrystallization yielded a product of [α]_{D}²⁵=+29.5° (c=1.0, H₂O) and a melting point of 210.0-214.5° C. Despite the use of optically pure(S)- or (R)-epichlorohydrin in the reaction with trimethylamine hydrochloride, the product was not obtained as optically pure and the optical purity of the crude product was only about 78%. Hence, a partially racemized product was obtained even when optically pure(S)-epichlorohydrin was reacted with trimethylamine hydrochloride under disclosed reaction conditions.

CN 102329243 discloses a continuous process in a tubular reactor for the reaction of(S)-epichlorohydrin dissolved in methanol with an aqueous solution of trimethylamine hydrochloride, wherein the ratio of the methanol solution of(S)-epichlorohydrin and the aqueous solution of trimethylamine hydrochloride is 1:0.92 (v/v). The product was obtained in a yield of 97% with a melting point of 190-191° C., which is much lower than 215° C. for a pure L-3-chloro-2-hydroxylpropyl trimethylammonium chloride. Hence, the method according CN 102329243 did not yield an optically pure product.

Since the reaction of L-3-chloro-2-hydroxylpropyl trimethylammonium chloride (II) with alkali cyanide to form L-carnitinenitrile chloride (III) and the subsequent hydrolysis of L-carnitinenitrile chloride to L-carnitine do not involve the chiral center, an optically impure L-3-chloro-2-hydroxylpropyl trimethylammonium chloride, produced according to prior art processes as an intermediated that is not isolated and purified, will result in the formation of an optically impure L-carnitinenitrile chloride. In order to produce optically pure L-carnitine, it has been known to recrystallize L-carnitinenitrile chloride to enrich its optical purity. This purification process is complicated and the yield of L-carnitinenitrile chloride is significantly reduced. Hence, the overall yield of L-carnitine is dramatically decreased.

It is the objective of the present invention to disclose a process for producing practically pure L-3-chloro-2-hydroxylpropyl trimethylammonium chloride from(S)-epichlorohydrin and its use to produce L-carnitine.

### SUMMARY OF THE INVENTION

The invention discloses an improved process for the production of L-carnitine by minimizing the racemization of(S)-epichlorohydrin during its reaction with trimethylamine hydrochloride to obtain optically pure L-3-chloro-2-hydroxylpropyl trimethylammonium chloride (II). The invention is accomplished by carrying out the reaction of(S)-epichlorohydrin with trimethylamine hydrochloride in an organic solvent.

### DETAILED DESCRIPTION OF THE INVENTION

The invention relates to an improved process for the production of L-carnitine. In particular, it discloses a process for producing practically pure L-3-chloro-2-hydroxylpropyl trimethylammonium chloride (II) by minimizing the racemization of optically active(S)-epichlorohydrin during its reaction with trimethylamine hydrochloride and the use of the practically pure L-3-chloro-2-hydroxylpropyl trimethylammonium chloride (II) to produce L-carnitine.

The invention is accomplished by a surprising and unexpected discovery that an optically pure L-3-chloro-2-hydroxylpropyl trimethylammonium chloride (II) can be produced if practically pure(S)-epichlorohydrin is reacted with anhydrous trimethylamine hydrochloride in an organic solvent. On the other hand, if the reaction is carried out in an aqueous solution, a partially racemized L-3-chloro-2-hydroxylpropyl trimethylammonium chloride (II) is obtained, even if the same practically pure(S)-epichlorohydrin is used.

Suitable organic solvents usable in the invention are selected from the group consisting of alkyl alcohols of C₁-C₁₂, ketones of C₃-C₁₂, nitriles of C₂-C₁₂, esters, ethers, amides, dialkyl carbonates, sulfones, halogenated alkanes, aliphatics, halogenated aromatics, and aromatics; wherein the alkyl is C₁-C₁₂ and wherein the halogen is fluorine, chlorine, bromine, or a mixture thereof. For example, suitable organic solvents can be selected from the group consisting of, but not limited to, methanol, ethanol, propanol, isopropanol, n-butanol, isobutanol, 2-butanol, tert-butanol, methoxyethanol, ethoxyethanol, propoxyethanol, butoxyethanol, ethylene glycol, propylene glycol, diethylene glycol, triethylene glycol, pentanol, isoamyl alcohol, hexanol, cyclohexanol, octanol, 2-ethylhexanol, acetone, butanone, pentanone, methyl isobutyl ketone, cyclohexanone, cyclopentanone, acetonitrile, propionitrile, butyronitrile, 3-methoxypropionitrile, 3-ethoxypropionitrile, 3-propoxyprionitrile, 3-butoxypropionitrile, benzonitrile, ethyl formate, propyl formate, butyl formate, propyl acetate, isopropyl acetate, isobutyl formate, cyclohexyl formate, pentyl formate, hexyl formate, methyl acetate, ethyl acetate, isobutyl acetate, butyl acetate, pentyl acetate, cyclohexyl acetate, methyl propionate, ethyl propionate, propyl propionate, methyl butyrate, ethyl butyrate, propyl butyrate, isopropyl butyrate, diethyl ether, dipropyl ether, diisopropyl ether, dibutyl ether, ethylene glycol dimethyl ether, ethylene glycol diethyl ether, ethylene glycol dibutyl ether, diethylene glycol dimethyl ether, diethyl glycol diethyl ether, tetrahydrofuran, 2-methyltetrahydrofuran, dioxane, dimethyl formamide, diethyl acetamide, diethyl formamide, diethyl acetamide, dipropyl formamide, dipropyl acetamide, diisopropyl formamide, diisopropyl acetamide, dibutyl formamide, dibutyl acetamide, diisobutyl formamide, diisobutyl acetamide, tetramethylurea, 1,3-dimethyl-2-imidazolidinone, dimethyl carbonate, diethyl carbonate, ethyl methyl carbonate, dimethyl sulfone, diethyl sulfone, tetramethylene sulfone, dichloromethane, chloroform, ethylene dichloride, trichloroethylene, tetrachloroethylene, hexanes, cyclohexane, methylcyclohexane, heptanes, octanes, benzene, toluene, xylenes, mesitylene, cumene, chlorobenzene, dichlorobenzenes, chlorotoluene, dichlorotoluene, nitrobenzene, nitrotoluene, trifluoromethylbenzene, and a mixture thereof.

If an organic solvent is water soluble, it may contain no more than 20% of water; preferably, not more than 10%; more preferably, not more than 5%.

(S)-epichlorohydrin can be produced by any method or obtained from any source. Preferably, (S)-epichlorohydrin is produced by the hydrolytic kinetic resolution of racemic epichlorohydrin by using a Jacobsen cobalt Salen catalyst or an analogue. Although practically pure(S)-epichlorohydrin is preferably used in the process according to the invention, there is no limit as to the purity of(S)-epichlorohydrin. When impure(S)-epichlorohydrin is used, a practically pure L-3-chloro-2-hydroxylpropyl trimethylammonium chloride (II) can still be obtained, but with reduced yield and the mother liquor solution cannot be recycled.

The molar ratio of chiral epichlorohydrin to trimethylamine hydrochloride is from 1:10 to 10:1. Preferably, the molar ratio is from 1:5 to 5:1. More preferably, the molar ratio is from 1:2 to 2:1. Most preferably the molar ratio is from 1.1:1 to 1:1.1.

The reaction temperature of chiral epichlorohydrin and trimethylamine hydrochloride in an organic solvent is from room temperature to 90° C. Preferably, the reaction temperature is from 30° C. to 80° C. More preferably, the reaction temperature is from 40° C. to 70° C.

The progress of the reaction of chiral epichlorohydrin and trimethylamine hydrochloride in an organic solvent can be monitored by using methods well known in the art. Once the epichlorohydrin is consumed, the reaction is deemed complete. Upon cooling, the product is obtained by using methods well known in the art.

It has been found that the mother liquor solution after the separation of L-3-chloro-2-hydroxylpropyl trimethylammonium chloride (II) can be recycled without adversely affecting the purity of the product for some solvents, for example, a lower alkyl alcohol.

L-3-chloro-2-hydroxylpropyltrimethylammonium chloride (II) can be reacted with a source of cyanide to produce L-carnitinenitrile chloride (III). A suitable source of cyanide is selected from the group consisting of alkali cyanide, alkaline earth metal cyanide, zinc cyanide, and a cyanohydrin; wherein the alkali is lithium, sodium, or potassium, and wherein the alkaline earth metal is magnesium, calcium, or barium.

It is particularly noted that L-carnitinenitrile chloride (III) is optically pure without further purification, if L-3-chloro-2-hydroxylpropyl trimethylammonium chloride (II) used in the reaction is produced according to the process of the invention. Since the reaction of L-3-chloro-2-hydroxylpropyl trimethylammonium chloride (II) with alkali cyanide does not involve the chiral center of the reactant and product, the optical purity of L-carnitinenitrile chloride is the same as the reactant. Hence, the process according to the invention ameliorates the necessity of complicated purification of L-carnitinenitrile chloride. This is an added advantage for the process according to the invention.

L-carnitinenitrile chloride can be hydrolyzed to L-carnitine by one of the known methods, for example by using hydrochloric acid. L-carnitine is then isolated from the hydrolysis solution by one of the known methods by using, for example, ion exchange resin or electrodialysis

The process according to the present invention can be carried out discontinuously, semi-continuously, or continuously.

### EXAMPLES

The following examples will illustrate the practice of this invention but are not intended to limit its scope.

### Comparative Example

In a round-bottom flask were added 140 mL of water and 47.8 g of trimethylamine hydrochloride to obtain a clear solution. After the solution was warmed up to 35° C., 46.3 g of(S)-epichlorohydrin was added dropwise in about 1 hour. The(S)-epichlorohydrin contained 99.6% (S)-epichlorohydrin and 0.4% (R)-epichlorohydrin by chiral gas chromatographic analysis. After being stirred for 6 hours at the same temperature, (S)-epichlorohydrin could not be detected and the reaction was deemed to be complete. The reaction solution was distilled to dryness under vacuum and 140 mL of ethanol was added to the residue and the suspension was stirred for 20 minutes. After crystalline solid was filtered, washed with ethanol, and dried to yield 64.3 g of the product with a specific rotation of [α]_{D}²⁵=−28.4° (c=1.0, H₂O).

The filtration mother liquor was distilled to dryness under vacuum. To the residue was added 20 mL of ethanol to yield a crystalline solid. The crystalline solid was filtered, washed with ethanol, and dried to obtain 10.3 g of the product with a specific rotation of [α]_{D}²⁵=−2.6° (c=1.0, H₂O).

### Example 1

To a round bottom flask were added 555 mL of anhydrous ethanol and 191.2 g of trimethylamine hydrochloride (2.0 mol) to obtain a suspension. The suspension was stirred and warmed up to 40° C. and then 185 g of(S)-epichlorohydrin (2.0 mol) was added dropwise over the course of 30 minutes. After reacting at the same temperature for 2 hours, epichlorohydrin was not detected by gas chromatography. The crystalline suspension was cooled to 5° C., stirred for 1 hour, and then filtered. After drying, 297.2 g of product was obtained. [α]_{D}²⁵=−27.9° (c=1.0, H₂O).

### Example 2

To a round bottom flask were added 140 mL of propanol and 47.7 g of trimethylamine hydrochloride (0.5 mol) to obtain a suspension. The suspension was stirred and warmed up to 40° C. and then 46.3 g of(S)-epichlorohydrin (0.5 mol) was added dropwise over the course of 30 minutes. After reacting at the same temperature for 2 hours, epichlorohydrin was not detected by gas chromatography. The crystalline suspension was cooled to 5° C., stirred for 1 hour, and then filtered. After drying, 75.6 g of product was obtained. [α]_{D}²⁵=−28.4° (c=1.0, H₂O).

### Example 3

To a round bottom flask were added 140 mL of isopropanol and 47.7 g of trimethylamine hydrochloride (0.5 mol) to obtain a suspension. The suspension was stirred and warmed up to 40° C. and then 46.3 g of(S)-epichlorohydrin (0.5 mol) was added dropwise over the course of 30 minutes. After reacting at the same temperature for 2 hours, epichlorohydrin was not detected by gas chromatography. The crystalline suspension was cooled to 5° C., stirred for 1 hour, and then filtered. After drying, 75.5 g of product was obtained. [α]_{D}²⁵=−27.5° (c=1.0, H₂O).

### Example 4

The following experiments demonstrated the recycling of mother liquor solution for the reaction of(S)-epichlorohydrin and trimethylamine hydrochloride. In each experiment, 1 mole each of(S)-epichlorohydrin and trimethylamine hydrochloride and 290 mL of ethanol and/or mother liquor solution were used in the reaction. The reactions were carried out at about 40° C. for about 2 hours to ensure that(S)-epichlorohydrin became absent in the reaction suspension. The molar yield of isolated product was calculated on the basis of(S)-epichlorohydrin.

| Experiment | No. | Molar Yield (%) | [α]_{D}²⁵ (c = 1.0, H₂O) |
|---|---|---|---|
| Initial Run | 0 | 75.1% | −28.5° |
| Recycling | 1 | 89.5% | −28.7° |
| | 2 | 78.8% | −28.6° |
| | 3 | 92.0% | −29.4° |
| | 4 | 91.0% | −28.8° |
| | 5 | 90.5% | −29.4° |
| | 6 | 90.4% | −29.0° |
| | 7 | 92.4% | −27.3° |
| | 8 | 90.9% | −29.6° |
| | 9 | 89.9% | −29.5° |
| | 10 | 94.1% | −28.7° |
| | 11 | 91.4% | −29.9° |
| | 12 | 92.4% | −29.9° |
| | 13 | 92.7% | −28.7° |
| | 14 | 91.8% | −28.8° |
| | 15 | 91.0% | −28.3° |

### Example 5

The following experiments demonstrated the use of selected organic solvents for the reaction of(S)-epichlorohydrin and trimethylamine hydrochloride. In each experiment, 1 mole each of(S)-epichlorohydrin and trimethylamine hydrochloride were used. And in each experiment, about 300 mL of solvent was used. The molar yield of isolated product was calculated on the basis of(S)-epichlorohydrin.

| | Reaction | Molar | |
|---|---|---|---|
| Solvent | Conditions | Yield (%) | [α]_{D}²⁵ (c = 1.0, H₂O) |
| Dimethyl formamide | 35° C.; 24 hrs | 66.4% | −28.3° |
| Diethyl acetamide | 35° C.; 24 hrs | 57.4% | −28.0° |
| o-Xylene | 60-70° C.; 6 hrs | 66.0% | −27.4° |
| N-Methylpyrrolidinone | 35° C., 18 hrs | 67.6% | −27.3° |
| Tetrahydrofuran | 50-65° C.; 20 hrs | 72.3% | −27.8° |
| Ethyl Acetate | 70° C.; 6 hrs | 76.1% | −28.9° |
| Acetone | 35°; 28 hrs | 77.6% | −27.6° |
| Acetonitrile | 35° C.; 20 hrs | 79.0% | −28.0° |
| Chloroform | 35° C.; 14 hrs | 79.9% | −27.3° |
| Dimethyl carbonate | 70° C., 6 hrs | 81.3% | −28.2° |
| Cyclohexane | 35° C.; 20 hrs | 84.1% | −28.6° |
| Dichloroethane | 40-60° C.; 3 hrs | 88.8% | −28.0° |

### Example 6

To a round bottom flask were added 50 mL of an aqueous solution containing 5.5 g of sodium cyanide (0.11 mol) and then 18.8 g of L-3-chloro-2-hydroxylpropyl trimethylammonium chloride (0.10 mol). After the mixture was stirred at 50° C. for 3 hrs, 5 mL of 30% hydrogen peroxide was added to destroy excess sodium cyanide. The solution was distilled to dryness under vacuum. To the residual solid was added 20 mL of 30% hydrochloric acid. The solution was stirred and heated to 95° C. for 3 hrs and then cooled to room temperature. The solution was then neutralized with aqueous solution of ammonia and applied to 500 mL of a strongly acidic resin bed. The absorbed L-carnitine was eluted with an aqueous solution of 3% ammonia. The L-carnitine fractions were combined and evaporated to dryness. The residue was dissolved in a minimal amount of anhydrous ethanol and L-carnitine was precipitated with acetone. [α]_{D}²⁵=−30.5° (c=10, H₂O).

It will be understood that the foregoing examples and explanation are for illustrative purposes only and that various modifications of the present invention will be self-evident to those skilled in the art. Such modifications are to be included within the spirit and purview of this application and the scope of the appended claims.

What is claimed is:

1. A process for the production of L-carnitine, comprising:
(a) reacting(S)-epichlorohydrin with trimethylamine hydrochloride in an organic solvent to obtain L-3-chloro-2-hydroxypropyl trimethylammonium chloride;
(b) reacting the L-3-chloro-2-hydroxypropyl trimethylammonium chloride of step (a) with a source of cyanide to yield L-carnitinenitrile chloride; and
(c) converting the L-carnitinenitrile chloride of step (b) to L-carnitine.

2. The process according to claim 1, wherein the organic solvent is selected from the group consisting of alkyl alcohols of C₁-C₁₂, ketones of C₃-C₁₂, nitriles of C₂-C₁₂, esters, ethers, amides, dialkyl carbonates, sulfones, halogenated alkanes, aliphatics, halogenated aromatics, and aromatics; wherein the alkyl is C₁-C₁₂ and wherein the halogen is fluoro, chloro, bromo, or a mixture thereof.

3. The process according to claim 1, wherein the organic solvent is methanol.

4. The process according to claim 1, wherein the organic solvent is ethanol.

5. The process according to claim 1, wherein the organic solvent is propanol, or isopropanol.

6. The process according to claim 1, wherein the organic solvent is n-butanol, isobutanol, 2-butanol, or tert-butanol.

7. The process according to claim 1, wherein the source of cyanide is selected from the group consisting of alkali cyanide, alkaline earth metal cyanide, zinc cyanide, and cyanohydrin; wherein alkali is lithium, sodium, potassium, or a mixture thereof; and wherein alkaline earth metal is magnesium, calcium, barium, or a mixture thereof.

8. The process according to claim 1, wherein the L-3-chloro-2-hydroxypropyl trimethylammonium chloride is produced in a cyclic process, comprising the steps of:
(1) mixing(S)-epichlorohydrin and trimethylammonium hydrochloride in an organic solvent to form L-3-chloro-2-hydroxypropyl trimethylammonium chloride;
(2) isolating the L-3-chloro-2-hydroxypropyl trimethylammonium chloride of step (1) to yield an organic mother liquor solution; and
(3) recycling the organic mother liquor solution of step (2) to step (1) as the organic solvent.

9. The process according to claim 8, wherein the organic solvent is selected from the group consisting of alkyl alcohols of C₁-C₁₂, ketones of C₃-C₁₂, nitriles of C₂-C₁₂, esters, ethers, amides, dialkyl carbonates, sulfones, halogenated alkanes, aliphatics, halogenated aromatics, and aromatics; wherein the alkyl is C₁-C₁₂ and wherein the halogen is fluoro, chloro, bromo, or a mixture thereof.
